(19) United States
(12) Patent Application Publication
(10) Pub. No.: US 2024/0296646 A1
(43) Pub. Date: Sep. 5, 2024

(54) IMAGE PROCESSING APPARATUS, IMAGE PROCESSING METHOD, AND STORAGE MEDIUM

(71) Applicant: OLYMPUS MEDICAL SYSTEMS CORP., Tokyo (JP)

(72) Inventors: Hiroshi TANAKA, Tokyo (JP); Takehito HAYAMI, Yokohama-shi (JP); Makoto KITAMURA, Tokyo (JP)

(73) Assignee: OLYMPUS MEDICAL SYSTEMS CORP., Tokyo (JP)

(21) Appl. No.: 18/662,403

(22) Filed: May 13, 2024

Related U.S. Application Data
(63) Continuation of application No. PCT/JP2021/047082, filed on Dec. 20, 2021.

(51) Int. Cl. G06V 10/24; G06T 7/00

(52) U.S. Cl. CPC G06V 10/247; G06T 7/0012; G06T 2207/10068

(57) ABSTRACT

An image processing apparatus includes a processor. After acquiring endoscope image information from an endoscope to generate an organ model, the processor continues acquiring the endoscope image information, specifies, based on the latest endoscope image information, a change site of the organ model already generated, corrects a shape of at least a part of the organ model including the change site, and outputs information on the organ model corrected.

### CROSS REFERENCE TO RELATED APPLICATION

This application is a continuation application of PCT/JP2021/047082 filed on Dec. 20, 2021, the entire contents of which are incorporated herein by this reference.

### BACKGROUND OF THE INVENTION

### 1. Field of the Invention

The present invention relates to an image processing apparatus, an image processing method, and a storage medium in which endoscope image information is acquired to generate an organ model.

### 2. Description of the Related Art

Endoscope examination is required to observe the entire area of an organ to be examined so as to prevent any lesion from being overlooked.

For example, U.S. Pat. No. 10,682,108 describes a technique of generating a three-dimensional organ model based on a two-dimensional endoscope image, using a DSO (direct sparse odometry) and a neural network. The three-dimensional organ model is used for, for example, identifying the position of an endoscope. Further, the three-dimensional organ model is also used for identifying an unobserved area by presenting a portion that is not visualized in the organ model (that is, an unobserved portion).

Some organs change in shape over time. Further, with the operation of withdrawing and inserting the endoscope, the shape of the organ and the position of the organ within a body occasionally change.

### SUMMARY OF THE INVENTION

An image processing apparatus according to one aspect of the present invention includes a processor, in which the processor is configured to: after acquiring endoscope image information from an endoscope to generate an organ model, continue acquiring the endoscope image information; specify, based on latest endoscope image information, a change site of the organ model already generated; correct a shape of at least a part of the organ model including the change site; and output information on the organ model corrected.

An image processing method according to one aspect of the present invention includes: after acquiring endoscope image information from an endoscope to generate an organ model, continuing acquiring the endoscope image information; specifying, based on latest endoscope image information, a change site of the organ model already generated; correcting a shape of at least a part of the organ model including the change site; and outputting information on the organ model corrected.

A storage medium according to one aspect of the present invention is a non-transitory storage medium that is readable by a computer and that stores a program, in which the program causes the computer to: after acquiring endoscope image information from an endoscope to generate an organ model, continue acquiring the endoscope image information, specify, based on latest endoscope image information, a change site of the organ model already generated, correct a shape of at least a part of the organ model including the change site, and output information on the organ model corrected.

### BRIEF DESCRIPTION OF THE DRAWINGS

FIG. 1 is a perspective view showing a configuration of an endoscope system in a first embodiment of the present invention;

FIG. 2 is a diagram mainly showing a structural and functional configuration of an image processing apparatus in the aforementioned first embodiment;

FIG. 3 is a block diagram showing an example of a configuration of the image processing apparatus of the aforementioned first embodiment when viewed as a structural unit;

FIG. 4 is a flowchart showing processing of the image processing apparatus of the aforementioned first embodiment;

FIG. 5 is a view for explaining generation of an organ model by an organ model generating section in the aforementioned first embodiment;

FIG. 6 is a chart showing an overall image of the organ model and an example of the organ model generated, corrected, and displayed in the aforementioned first embodiment;

FIG. 7 is a view for explaining an example of detecting a change in the organ model based on feature points in the aforementioned first embodiment;

FIG. 8 is a flowchart showing processing of an image processing apparatus of a second embodiment in the present invention;

FIG. 9 is a chart showing a state of estimating a change amount between organ models at different times in the aforementioned second embodiment;

FIG. 10 is a view showing an example of correcting a shape of the organ model based on the change amount in the aforementioned second embodiment;

FIG. 11 is a flowchart showing processing of estimating the change amount in the organ model in step S12 of FIG. 8 in the aforementioned second embodiment;

FIG. 12 is a chart showing a state in which the change in the organ model includes expansion, rotation, and movement in the aforementioned second embodiment;

FIG. 13 is a flowchart showing processing of detecting an expansion and reduction amount in step S21 of FIG. 11 in the aforementioned second embodiment;

FIG. 14 is a chart for explaining the processing of detecting the expansion and reduction amount in the aforementioned second embodiment;

FIG. 15 is a flowchart showing processing of detecting a rotation amount in step S22 of FIG. 11 in the aforementioned second embodiment;

FIG. 16 is a chart for explaining the processing of detecting the rotation amount in the aforementioned second embodiment;

FIG. 17 is a flowchart showing processing of detecting an extension and contraction amount in step S23 of FIG. 11 in the aforementioned second embodiment;

FIG. 18 is a chart for explaining the processing of detecting the extension and contraction amount in the aforementioned second embodiment;

FIG. 19 is a flowchart showing processing of detecting a moving amount in step S24 of FIG. 11 in the aforementioned second embodiment;

FIG. 20 is a chart for explaining an example of a method for correcting the shape of the organ model in step S3A of FIG. 8 in the aforementioned second embodiment;

FIG. 21 is a flowchart showing processing of an image processing apparatus of a third embodiment in the present invention;

FIG. 22 is a flowchart showing processing of estimating a change amount of a fold in step S12B of FIG. 21 in the aforementioned third embodiment;

FIG. 23 is a chart for explaining processing of detecting presence or absence of passing of the fold in the aforementioned third embodiment;

FIG. 24 is a chart for explaining a state of associating the fold in the endoscope image and the fold in an organ model in the aforementioned third embodiment;

FIG. 25 is a chart for explaining a state of detecting a change amount of an identical fold in the aforementioned third embodiment;

FIG. 26 is a flowchart showing processing of detecting the change amount of the identical fold in step S73 of FIG. 22 in the aforementioned third embodiment;

FIG. 27 is a flowchart showing another processing example of detecting an expansion and reduction amount of a diameter in step S81 of FIG. 26 in the aforementioned third embodiment;

FIG. 28 is a chart for explaining the other processing example of detecting the expansion and reduction amount of the diameter in the aforementioned third embodiment;

FIG. 29 is a graph for explaining an example of a method for correcting the expansion and reduction amount of the diameter of the organ model in the aforementioned third embodiment;

FIG. 30 is a view for explaining an example of correcting the expansion and reduction amount of the diameter of the organ model in a correction range in the aforementioned third embodiment;

FIG. 31 is a graph for explaining an example of a method for correcting a rotation amount of the organ model in the aforementioned third embodiment;

FIG. 32 is a chart for explaining processing of detecting an extension and contraction amount in step S83 of FIG. 26 in the aforementioned third embodiment;

FIG. 33 is a graph for explaining an example of a method for correcting the extension and contraction amount of the organ model in the aforementioned third embodiment;

FIG. 34 is a view for explaining an example of correcting the extension and contraction amount of the organ model in the aforementioned third embodiment;

FIG. 35 is a flowchart for explaining processing of detecting a moving amount in step S84 of FIG. 26 in the aforementioned third embodiment;

FIG. 36 is a view showing an example of detecting an identical fold in an existing organ model and a new organ model for determining movement of the organ in the aforementioned third embodiment;

FIG. 37 is a view for explaining a method for correcting the shape of the organ model in accordance with the movement of the organ in the aforementioned third embodiment;

FIG. 38 is a graph for explaining the method for correcting the shape of the organ model in accordance with the movement of the organ in the aforementioned third embodiment; and

FIG. 39 is a chart showing an example of displaying the organ model and an unobserved area in the aforementioned third embodiment.

### DETAILED DESCRIPTION OF THE PREFERRED EMBODIMENTS

Hereinafter, embodiments of the present invention will be described with reference to the drawings. However, the present invention is not limited to the embodiments described below.

Note that in the descriptions of the drawings, the same or corresponding elements are assigned the same reference signs, as appropriate. It should be noted that the drawings are schematic illustrations and that the length relation among the elements, the length ratio among the elements, the number of the elements, and the like in one drawing are different from the actual length relation, length ratio, number, and the like, for the sake of simple explanation. Moreover, portions having different length relations and ratios among a plurality of drawings are included in some cases.

### First Embodiment

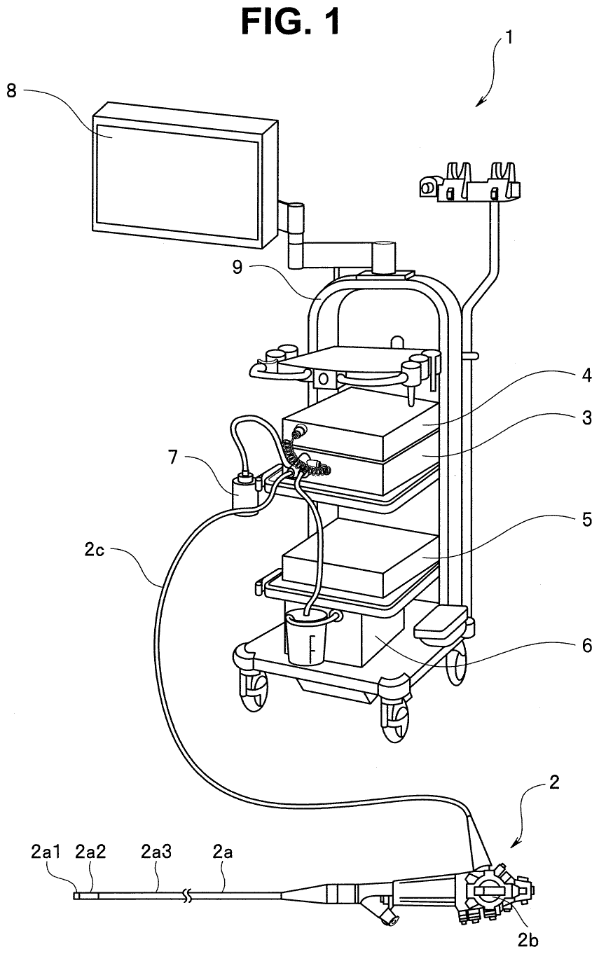

FIG. 1 to FIG. 7 show a first embodiment of the present invention, and FIG. 1 is a perspective view showing a configuration of an endoscope system 1 in the first embodiment.

The endoscope system 1 includes, for example, an endoscope 2, a light source apparatus 3, an image processing apparatus 4, a distal end position detecting apparatus 5, a suction pump 6, a liquid feeding tank 7, and a monitor 8. The abovementioned components other than the endoscope 2 are placed on or fixed to a cart 9 as shown in FIG. 1. The endoscope system 1 is disposed, for example, in an examination room where a subject is examined and treated. The light source apparatus 3 and the image processing apparatus 4 may be separate bodies or may be integrated as an image processing apparatus with an integrated light source. For the distal end position detecting apparatus 5, a technique of identifying a position of a distal end of the endoscope by generating a magnetic field can be adopted, for example. As the distal end position detecting apparatus 5, a publicly-known insertion shape detecting device (UPD) may also be adopted.

The endoscope 2 includes an insertion portion 2a, an operation portion 2b, and a universal cable 2c.

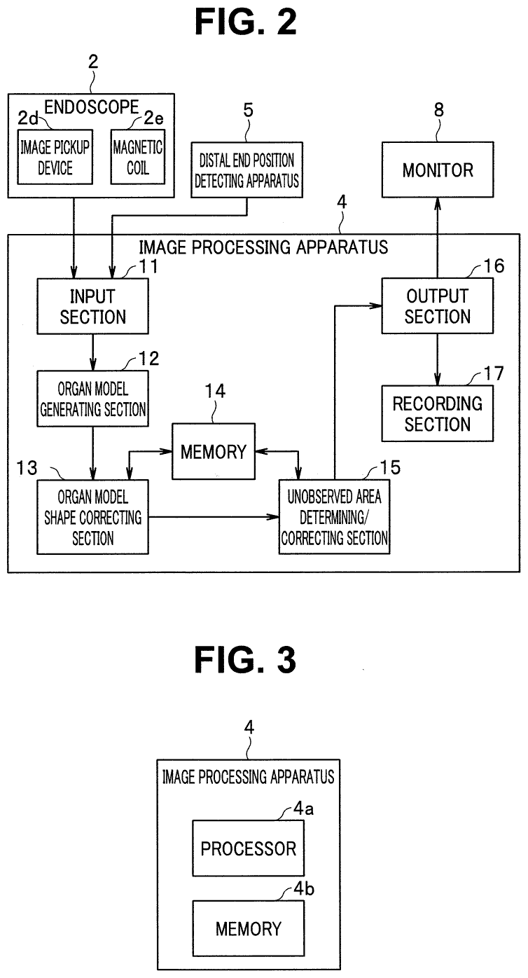

The insertion portion 2a is a site to be inserted into a subject, and includes a distal end portion 2a1, a bending portion 2a2, and a flexible tube portion 2a3 in sequence from a distal end side toward a proximal end side. In the distal end portion 2a1, an image pickup unit including an image pickup optical system and an image pickup device 2d (see FIG. 2), a magnetic coil 2e (see FIG. 2), a distal end portion of a light guide, an opening on a distal end side of a treatment instrument channel, and the like are disposed.

The operation portion 2b is disposed on a proximal end side of the insertion portion 2a and is a site with which various operations are performed by hands.

The universal cable 2c extends from the operation portion 2b, for example, and is a connection cable for connecting the endoscope 2 to the light source apparatus 3, the image processing apparatus 4, the suction pump 6, and the liquid feeding tank 7.

The light guide, a signal cable, the treatment instrument channel also serving as a suction channel, and an air/liquid feeding channel are inserted through the inside of the insertion portion 2a, the operation portion 2b, and the universal cable 2c of the endoscope 2.

A connector provided at an extension end of the universal cable 2c is connected to the light source apparatus 3. A cable extending from the connector is connected to the image processing apparatus 4. Therefore, the endoscope 2 is connected to the light source apparatus 3 and the image processing apparatus 4.

The light source apparatus 3 includes, as a light source, a light emitting device, such as an LED (light emitting diode) light source, a laser light source, or a xenon light source. With the connector connected to the light source apparatus 3, transmission of illumination light to the light guide is enabled.

The illumination light made incident on a proximal end surface of the light guide from the light source apparatus 3 is transmitted through the light guide to be irradiated toward a subject from a distal end surface of the light guide disposed in the distal end portion 2a1 of the insertion portion 2a.

The suction channel and the air/liquid feeding channel are respectively connected to the suction pump 6 and the liquid feeding tank 7, for example, via the light source apparatus 3. Therefore, with the connector connected to the light source apparatus 3, suction in the suction channel by the suction pump 6, liquid feeding from the liquid feeding tank 7 via the air/liquid feeding channel, and air feeding via the air/liquid feeding channel are enabled.

The suction pump 6 is used to suck a liquid or the like from a subject.

The liquid feeding tank 7 is a tank for storing a liquid such as a physiological salt solution. Pressurized air is fed from an air/liquid feeding pump in the light source apparatus 3 to the liquid feeding tank 7 so that the liquid inside the liquid feeding tank 7 is fed to the air/liquid feeding channel.

The distal end position detecting apparatus 5 detects, by means of a magnetic sensor (position detecting sensor), the magnetism generated from one or more magnetic coils 2e (see FIG. 2) provided in the insertion portion 2a so as to detect the shape of the insertion portion 2a. The position and the pose of the distal end portion 2a1 of the insertion portion 2a are detected by the distal end position detecting apparatus 5.

The image processing apparatus 4 transmits a drive signal for driving the image pickup device 2d (see FIG. 2) via the signal cable. An image pickup signal outputted from the image pickup device 2d is transmitted to the image processing apparatus 4 via the signal cable.

The image processing apparatus 4 performs image processing on the image pickup signal acquired by the image pickup device 2d, and generates and outputs a displayable image signal. Further, the position information on the distal end portion 2a1 of the insertion portion 2a acquired by the distal end position detecting apparatus 5 is inputted to the image processing apparatus 4. Note that the image processing apparatus 4 may control not only the endoscope 2, but the whole endoscope system 1 including the light source apparatus 3, the distal end position detecting apparatus 5, the suction pump 6, the monitor 8, and the like.

The monitor 8 displays an image including an endoscope image, in accordance with the image signal outputted from the image processing apparatus 4.

FIG. 2 is a diagram mainly showing a structural and functional configuration of the image processing apparatus in the first embodiment. Note that in FIG. 2, illustrations of the light source apparatus 3, the suction pump 6, the liquid feeding tank 7, and the like are omitted.

The endoscope 2 is configured as an electronic endoscope and includes, in the distal end portion 2a1 of the insertion portion 2a, the image pickup device 2d and the magnetic coil 2e.

The image pickup device 2d picks up an optical image of a subject that is formed by the image pickup optical system and generates an image pickup signal. The image pickup device 2d picks up images by frame unit, for example, and generates image pickup signals regarding the images of a plurality of frames in a chronological order. The generated image pickup signals are sequentially outputted to the image processing apparatus 4 via the signal cable connected to the image pickup device 2d.

The position and the pose of the distal end portion 2a1 of the insertion portion 2a that are detected by the distal end position detecting apparatus 5 are outputted to the image processing apparatus 4 based on the magnetism generated by the magnetic coil 2e.

The image processing apparatus 4 includes an input section 11, an organ model generating section 12, an organ model shape correcting section 13, a memory 14, an unobserved area determining/correcting section 15, an output section 16, and a recording section 17.

The input section 11 receives the image pickup signal from the image pickup device 2d and the information on the position and the pose of the distal end portion 2a1 of the insertion portion 2a from the distal end position detecting apparatus 5.

The organ model generating section 12 acquires, from the input section 11, endoscope image information (hereinafter, referred to as an endoscope image, as appropriate) regarding the image pickup signal. Then, the organ model generating section 12 detects, from the endoscope image, the position and the pose of the distal end portion 2a1 of the insertion portion 2a. Further, the organ model generating section 12 acquires, as necessary, the information on the position and the pose of the distal end portion 2a1 of the insertion portion 2a from the distal end position detecting apparatus 5 via the input section 11. Furthermore, the organ model generating section 12 generates a three-dimensional organ model based on the position and the pose of the distal end portion 2a1 and the endoscope image.

The organ model shape correcting section 13 corrects the shape of the organ model (existing organ model) already generated in the past, based on the latest endoscope image.

The memory 14 stores the corrected organ model.

The unobserved area determining/correcting section 15 determines an unobserved area in the corrected organ model and corrects the position and the shape of the unobserved area in accordance with the corrected organ model. The position and the shape of the unobserved area are stored in the memory 14, as necessary.

The output section 16 outputs the information on the corrected organ model. Further, the output section 16 also outputs the information on the unobserved area, as necessary.

The recording section 17 stores, in a nonvolatile manner, the endoscope image information that was subjected to the image processing by the image processing apparatus 4 and outputted from the output section 16. Note that the recording section 17 may be a recording device provided outside the image processing apparatus 4.

The information (further information on the unobserved area, as necessary) on the organ model outputted from the output section 16 is displayed on the monitor 8, as an organ model image, together with the endoscope image, for example.

FIG. 2 shows the functional configuration of each hardware of the image processing apparatus 4, and FIG. 3 is a block diagram showing an example of a configuration of the image processing apparatus 4 of the first embodiment when viewed as a structural unit.

As shown in FIG. 3, the image processing apparatus 4 includes a processor 4a including hardware and a memory 4b. The processor 4a includes, for example, an ASIC (application specific integrated circuit) including a CPU (central processing unit) and the like, an FPGA (field programmable gate array), or a GPU (graphics processing unit).

The memory 4b includes the memory 14 of FIG. 2 and includes, for example, a volatile storage medium, such as a RAM (random access memory), and a nonvolatile storage medium, such as a ROM (read only memory) (or an EEPROM (electrically erasable programmable read-only memory)). The RAM temporarily stores various information, such as an image of a processing target, processing parameters at the time of execution, and set values by a user that are externally inputted. The ROM stores, in a nonvolatile manner, various information such as processing programs (computer programs), specified values of processing parameters, and set values by a user that should be kept stored even when the power of the endoscope system 1 is turned off.

The processor 4a shown in FIG. 3 reads and executes the processing programs stored in the memory 4b so that various functions of the image processing apparatus 4 as shown in FIG. 1 are achieved. However, the configuration may be made such that all or part of the various functions of the image processing apparatus 4 may be achieved by a dedicated electronic circuit.

Herein, the example of storing the processing programs in the memory 4b is described, but the processing programs (or at least part of the processing programs) may be stored in a removable storage medium, such as a flexible disc, a CD-ROM (compact disc read only memory), a DVD (digital versatile disc), or a Blu-ray disc, a storage medium, such as a hard disc drive or an SSD (solid state drive), a storage medium on a cloud, and the like. In this case, it is only necessary that the processing programs are read from the external storage medium and caused to be stored in the memory 4b, and the processor 4a executes the processing programs.

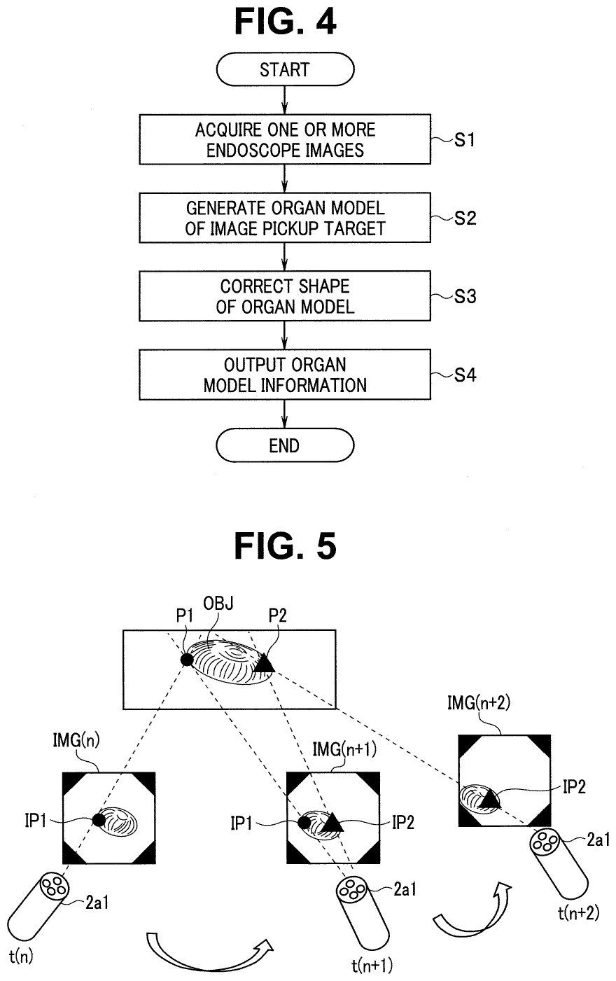

FIG. 4 is a flowchart showing processing of the image processing apparatus 4 of the first embodiment.

When the power of the endoscope system 1 is turned on and the endoscope 2 starts picking up images and outputting image pickup signals, the image processing apparatus 4 executes the processing shown in FIG. 4 each time the image pickup signals (endoscope image information), for example, by one frame are inputted.

The image processing apparatus 4 acquires the latest one or more endoscope images by means of the input section 11 (step S1).

The organ model generating section 12 generates an organ model of an image pickup target based on the acquired one or more endoscope images (step S2). To generate a three-dimensional organ model, it is preferable that a plurality of endoscope images picked up at different positions should be used, but with the use of AI (artificial intelligence), it is also possible to generate a three-dimensional organ model from one endoscope image.

The organ model shape correcting section 13 corrects the shape of the organ model (existing organ model) already generated in the past, based on the latest endoscope image (step S3).

Herein, when the endoscope image acquired in step S2 is the first image after the endoscope 2 has started picking up images, there is no organ model already generated, and thus, the organ model shape correcting section 13 does not perform correction and causes the memory 14 to store the organ model acquired from the organ model generating section 12.

When the endoscope image acquired in step S2 is the second image or the subsequent images after the endoscope 2 has started picking up images, the organ model shape correcting section 13 acquires, from the organ model generating section 12, a new organ model generated based on the latest endoscope image and also acquires the latest endoscope image, as necessary. Further, the organ model shape correcting section 13 acquires the existing organ model from the memory 14. Then, the organ model shape correcting section 13 determines, based on at least one of the latest endoscope image or the new organ model, whether the existing organ model needs to be corrected. When it is determined that correction is necessary, the organ model shape correcting section 13 corrects the existing organ model based on the new organ model. The organ model shape correcting section 13 causes the memory 14 to store the corrected organ model.

The output section 16 outputs the information on the organ model corrected by the organ model shape correcting section 13 to the monitor 8 (step S4). Thus, the organ model image is displayed on the monitor 8.

As described above, the processing shown in FIG. 4 is executed each time the latest endoscope image is acquired. Therefore, the user can check, on the monitor 8, the organ model generated based on the latest endoscope image information and matching the current organ.

FIG. 5 is a view for explaining generation of an organ model by the organ model generating section 12 in the first embodiment.

The organ model generating section 12 generates a 3D organ model by, for example, visual SLAM (visual simultaneous localization and mapping). The organ model generating section 12 may estimate the position and the pose of the distal end portion 2a1 of the insertion portion 2a by processing of the visual SLAM or may use the information inputted from the distal end position detecting apparatus 5.

The organ model generating section 12 first performs initialization in generating a three-dimensional organ model. In the initialization, the internal parameters of the endoscope 2 are assumed to be known through calibration. As the initialization, the organ model generating section 12 estimates the own position and the three-dimensional position of the endoscope 2 using, for example, an SfM (structure from motion). Herein, the SLAM inputs, for example, temporally continuous dynamic images or the like on the premise of real-time performance, while the SfM inputs a plurality of images that are not on the premise of real-time performance.

It is assumed that after performing the initialization, the endoscope 2 has moved. FIG. 5 shows a state in which the position of the distal end portion 2a1 of the insertion portion 2a moves as the time proceeds from t(n), t(n+1) to t(n+2).

At this time, the organ model generating section 12 searches for a corresponding point among the endoscope images of the plurality of frames.

Specifically, in the example of FIG. 5, among an endoscope image IMG(n) picked up at time t(n), an endoscope image IMG(n+1) picked up at time t(n+1) after time t(n), and an endoscope image IMG(n+2) picked up at time t(n+2) after time t(n+1), a corresponding point is searched for.

For example, an image point IP1 corresponding to a point P1 in an organ OBJ of a subject is located in the endoscope image IMG(n) and the endoscope image IMG(n+1), but is not located in the endoscope image IMG(n+2), and an image point IP2 corresponding to a point P2 in the organ OBJ of the subject is not located in the endoscope image IMG(n), but is located in the endoscope image IMG(n+1) and the endoscope image IMG(n+2).

Next, the organ model generating section 12 estimates (tracks) the position and the pose of the endoscope 2. The problem in estimating the position and the pose of the endoscope 2 (more commonly, a camera) is estimation of the position and the pose of the camera (endoscope 2 in the present embodiment) from three-dimensional coordinates of a point n in the world coordinate system and coordinates of the image where the point n is observed, which is a so-called PnP problem.

First, the organ model generating section 12 estimates the pose of the endoscope 2 based on a plurality of points, the three-dimensional positions of which are known, and the positions of the plurality of points on the image.

Subsequently, the organ model generating section 12 resisters (maps) the points on a 3D map. In other words, a common point appearing on the plurality of endoscope images acquired by the endoscope 2, the pose of which has become known, can be associated, so that the three-dimensional position of the point can be identified (triangulation).

Thereafter, the organ model generating section 12 repeats the aforementioned tracking and mapping, so that the three-dimensional position of any point on the endoscope image can be recognized, and the organ model is generated.

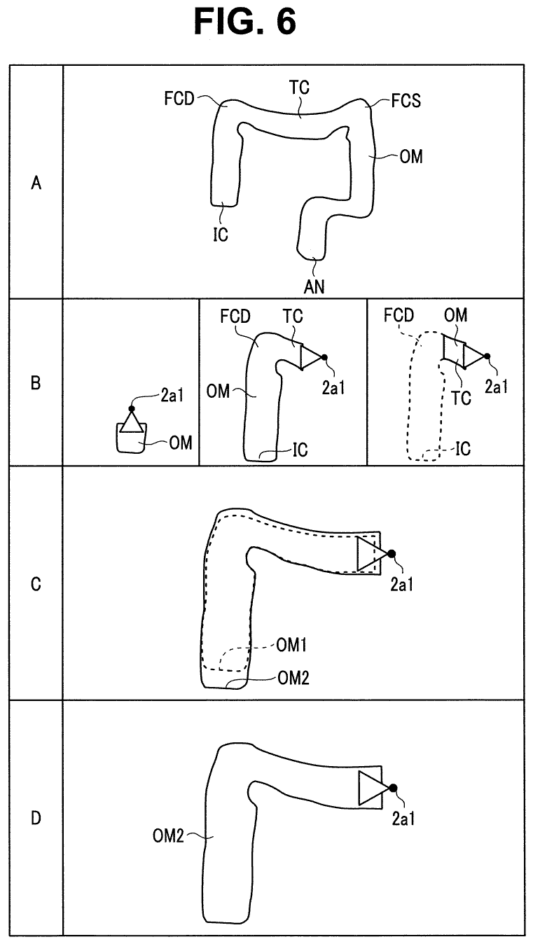

FIG. 6 is a chart showing an overall image of the organ model and an example of the organ model generated, corrected, and displayed in the first embodiment.

Column A of FIG. 6 shows an overall image of an organ model OM. Herein, as an example of the organ model OM, an organ model of an intestine, specifically, a colon is illustrated, but the organ model is not limited to the illustrated model. In Column A of FIG. 6, IC represents an intestinal cecum, AN represents an anal, FCD represents a hepatic flexure (right colonic flexure), FCS represents a splenic flexure (left colonic flexure), and TC represents a transverse colon.

Column B of FIG. 6 shows a state of the organ model OM generated while the insertion portion 2a of the endoscope 2 moves from the intestinal cecum IC side through the hepatic flexure FCD toward the splenic flexure FCS side. Note that a triangle in each of Columns B to D of FIG. 6 shows a state of an angular field of view when a subject is observed from the distal end portion 2a1 of the insertion portion 2a. In Column left B of FIG. 6, the organ model OM near the intestinal cecum IC is generated.

In Column center B of FIG. 6, since the insertion portion 2a passes the hepatic flexure FCD and is moving toward the transverse colon TC side, the organ model OM from the intestinal cecum IC to the hepatic flexure FCD and a portion of the transverse colon TC is generated.

Column right B of FIG. 6 shows a modification of Column center B. It is assumed that of the organ model OM in Column center B of FIG. 6, a portion denoted by a dotted line in Column right B has no unobserved area. At this time, the portion of the existing organ model denoted by the dotted line may not be retained in the memory 14 or may not be displayed on the monitor 8.

In Column C of FIG. 6, a portion denoted by a broken line represents an organ model OM1 before correction and a portion denoted by a solid line represents an organ model OM2 after correction. When the organ model OM2 after correction is generated, the organ model OM1 before correction is deleted.

As shown in Column D of FIG. 6, the organ model OM2 after correction is displayed on the monitor 8.

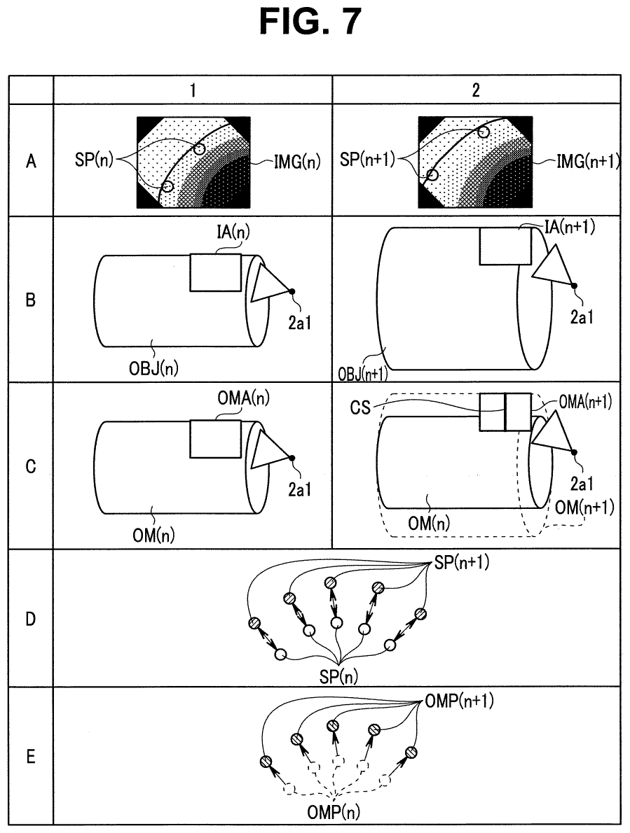

FIG. 7 is a view for explaining an example of detecting a change in the organ model based on feature points in the first embodiment. The feature point is one of specific targets included in the endoscope image information.

Column A1 of FIG. 7 shows a state of the endoscope image IMG(n) picked up at time t(n). Column A2 of FIG. 7 shows a state of the endoscope image IMG(n+1) picked up at time t(n+1). A plurality of feature points SP(n) in the endoscope image IMG(n) and a plurality of feature points SP(n+1) in the endoscope image IMG(n+1) are corresponding identical feature points (points having identical feature values).

Column B1 of FIG. 7 shows an organ OBJ(n) of a subject at time t(n) and an image pickup area IA(n) of the endoscope 2. Column B2 of FIG. 7 shows an organ OBJ(n+1) of the subject at time t(n+1) and an image pickup area IA(n+1) of the endoscope 2. When Column B1 and Column B2 are compared, a lumen diameter of the organ OBJ(n+1) of the subject at time t(n+1) is enlarged as compared to a lumen diameter of the organ OBJ(n) of the subject at time t(n).

Column C1 of FIG. 7 shows an organ model OM(n) at time t(n) and an organ model area OMA(n) corresponding to the image pickup area IA(n). Column C2 of FIG. 7 shows an organ model OM(n+1) at time t(n+1) and an organ model area OMA(n+1) corresponding to the image pickup area IA(n+1), in comparison with the organ model OM(n).

Column D of FIG. 7 shows a state of detecting, using a feature value, a luminance value, a luminance gradient value, and the like, the plurality of feature points SP(n) in the organ model area OMA(n) and the plurality of feature points SP(n+1) in the organ model area OMA(n+1) that correspond to the plurality of feature points SP(n) on a cross-section CS shown in Column C2 of FIG. 7. Since the lumen diameter is enlarged at time t(n+1), the feature points SP(n+1) are the points as the feature points SP(n) having moved in such a manner as expanding in a radial direction.

Column E of FIG. 7 shows a state in which of the corresponding points at time t(n) and at time t(n+1), points OMP(n) on the existing organ model OM(n) are deleted and the new organ model OM(n+1) is generated from points OMP(n+1) acquired from the latest endoscope image.

According to such a first embodiment, since the shape of the organ model already generated is corrected, the organ model matching the current shape of the organ can be generated. Further, since the points OMP(n) on the existing organ model OM(n) are deleted, a plurality of organ models are not generated for the same area, so that the organ model is an appropriate model.

### Second Embodiment

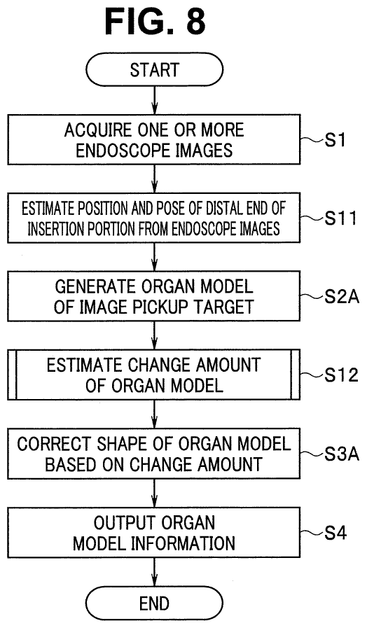

FIG. 8 to FIG. 20 show a second embodiment of the present invention, and FIG. 8 is a flowchart showing processing of the image processing apparatus 4 of the second embodiment. In the second embodiment, for the portions that are the same as the portions of the first embodiment, the same reference signs are assigned and the descriptions will be omitted, as appropriate, and different points will be mainly described.

Upon starting the processing shown in FIG. 8, the image processing apparatus 4 performs the processing of step S1 to acquire the latest one or more endoscope images and the organ model generating section 12 estimates, from the acquired endoscope images, the position and the pose of the distal end portion 2a1 of the insertion portion 2a (step S11).

Next, the organ model generating section 12 generates an organ model of an image pickup target based on the estimated position and pose of the distal end portion 2a1 of the insertion portion 2a (step S2A).

Then, the organ model shape correcting section 13 specifies a change site in the current organ model (new organ model) of the image pickup target generated in step S2A relative to the organ model (existing organ model) already generated in the past and estimates a change amount of the change site (step S12). The estimation of the change amount is performed, for example, based on the change amount of a corresponding point (such as a feature point) on the cross-section between the existing organ model and the new organ model. For example, when the processing shown in FIG. 4 is executed each time the endoscope image information is inputted by one frame, calculation of the change amount is also performed by one frame.

Subsequently, the organ model shape correcting section 13 corrects the shape of the existing organ model based on the estimated change amount of the organ model (step S3A).

Thereafter, the processing of step S4 is performed to output the information on the corrected organ model to the monitor 8 or the like.

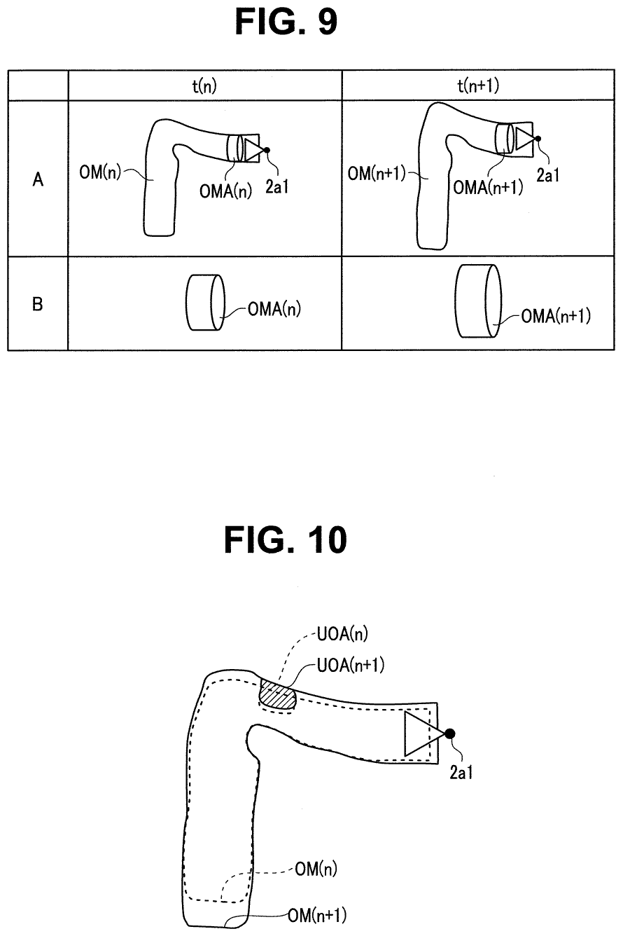

FIG. 9 is a chart showing a state of estimating a change amount between organ models at different times in the second embodiment.

Note that the overall image of the presumed organ model is the image shown in Column A of FIG. 6. When there is no unobserved area, the portion of the organ model denoted by the dotted line in Column B of FIG. 6 may not be retained in the memory 14 or may not be displayed on the monitor 8, which is the same as in the first embodiment.

Column left A of FIG. 9 shows the organ model area OMA(n) of a target for detection of the change amount in the organ model OM(n) at time t(n). Column right A of FIG. 9 shows the organ model area OMA(n+1) of the target for detection of the change amount in the organ model OM(n+1) at time t(n+1).

In the organ model area OMA(n+1) at time t(n+1) shown in Column right B of FIG. 9, the lumen diameter is enlarged by, for example, an appropriate scalar multiple as compared to the lumen diameter of the organ model area OMA(n) at time t(n) shown in Column left B of FIG. 9.

FIG. 10 is a view showing an example of correcting the shape of the organ model based on the change amount in the second embodiment.

The organ model OM(n) at time t(n) in the past is corrected to the organ model OM(n+1) at time t(n+1) at present.

At this time, the unobserved area determining/correcting section 15 determines an unobserved area UOA(n+1) in the organ model OM(n+1) after correction. For example, the unobserved area determining/correcting section 15 determines whether an unobserved area UOA(n) has turned into an observed area, and determines whether the unobserved area UOA(n) has moved to the unobserved area UOA(n+1) when the unobserved area UOA(n) has not turned into the observed area, and also determines whether the new unobserved area UOA(n+1) has been generated or the like.

Then, the unobserved area determining/correcting section 15 superposes the generated unobserved area UOA(n+1) on the organ model OM(n+1) after correction to be outputted to the output section 16. Thus, an organ model image of the new organ model OM(n+1) with the position or the shape corrected or with the new unobserved area UOA(n+1) superposed is displayed on the monitor 8, together with the endoscope image, for example. The unobserved area determining/correcting section 15 may retain the unobserved area UOA(n+1) in the memory 14.

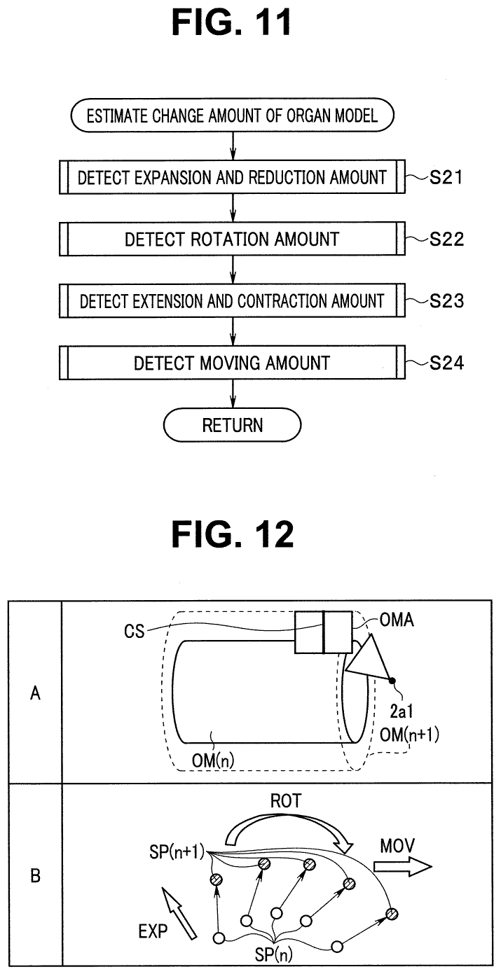

FIG. 11 is a flowchart showing processing of estimating the change amount in the organ model in step S12 of FIG. 8 in the second embodiment.

The estimation of the change amount of the organ model by the organ model shape correcting section 13 is performed, for example, by detecting an expansion and reduction amount of the lumen diameter in the new organ model relative to the existing organ model (step S21), detecting a rotation amount about the center axis (lumen axis) of the lumen (step S22), detecting an extension and contraction amount of the lumen along the lumen axis (step S23), and detecting a moving amount of the lumen in a subject (step S24). Note that FIG. 11 shows an example of the detecting order, but the detecting order is not limited to the illustrated detecting order.

FIG. 12 is a chart showing a state in which the change in the organ model includes expansion, rotation, and movement in the second embodiment.

Column B of FIG. 12 shows a state of the organ models OM(n), OM(n+1) at different times t(n), t(n+1) that are shown in Column A of FIG. 12, when the cross-section CS perpendicular to the lumen axis of the organ model area OMA is taken.

The change in the organ model OM from the plurality of feature points SP(n) to the plurality of feature points SP(n+1) includes, for example, expansion EXP of the lumen diameter, rotation ROT of the lumen about the lumen axis, and movement MOV of the lumen in a subject.

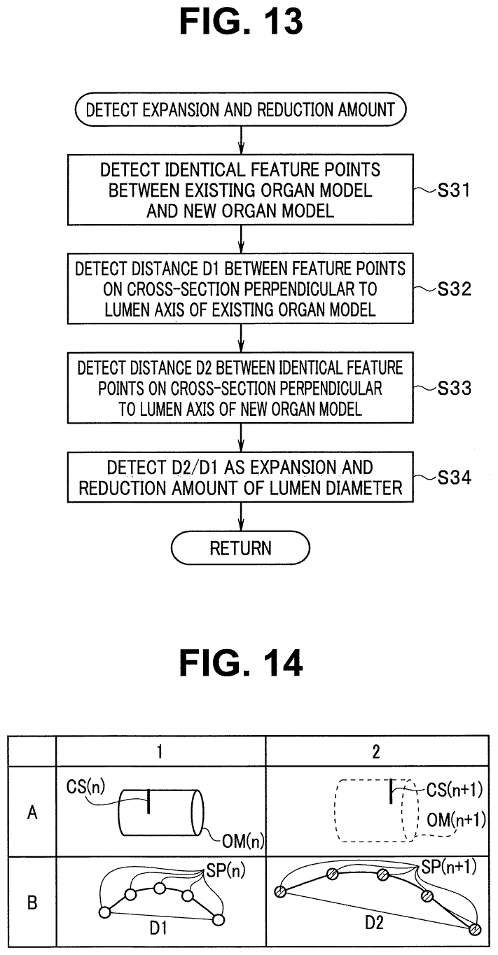

FIG. 13 is a flowchart showing processing of detecting an expansion and reduction amount in step S21 of FIG. 11 in the second embodiment. FIG. 14 is a chart for explaining the processing of detecting the expansion and reduction amount in the second embodiment.

Upon starting the processing of detecting the expansion and reduction amount shown in FIG. 13, the organ model shape correcting section 13 detects the feature points SP(n) of the existing organ model OM(n) read from the memory 14 and the feature points SP(n+1), which correspond to the feature points SP(n), in the new organ model OM(n+1) generated by the organ model generating section 12 based on the latest endoscope image (step S31).

Next, the organ model shape correcting section 13 detects a distance D1 between two specific feature points SP(n) on the cross-section CS(n) perpendicular to the lumen axis of the existing organ model OM(n), as shown in Column A1 and Column B1 of FIG. 14 (step S32).

Further, the organ model shape correcting section 13 detects a distance D2 between two specific feature points SP(n+1), which correspond to the feature points SP(n), between which the distance D1 was detected, on the cross-section CS(n+1) perpendicular to the lumen axis of the new organ model OM(n+1), as shown in Column A2 and Column B2 of FIG. 14 (step S33).

Then, the organ model shape correcting section 13 sets the ratio of the distance D2 to the distance D1 (D2/D1) as the expansion and reduction amount of the lumen diameter (step S34), and returns to the processing of FIG. 11. Note that when the ratio (D2/D1) is greater than 1, the lumen diameter is expanded, while the ratio (D2/D1) is smaller than 1, the lumen diameter is reduced.

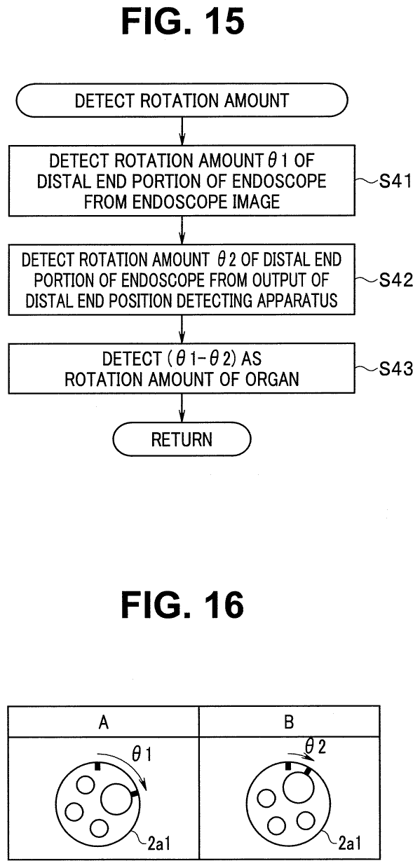

FIG. 15 is a flowchart showing processing of detecting a rotation amount in step S22 of FIG. 11 in the second embodiment. FIG. 16 is a chart for explaining the processing of detecting the rotation amount in the second embodiment.

The organ model shape correcting section 13 performs image estimation through, for example, the SLAM processing, based on the endoscope images picked up at different times that are acquired from the input section 11 via the organ model generating section 12, and detects a first rotation amount θ1 of the distal end portion 2a1 of the insertion portion 2a, as shown in Column A of FIG. 16 (step S41). For example, when the rotation amount, which is detected based on the specific target (such as feature points), in the plurality of pieces of endoscope image information picked up at different times is −θ1, the organ model shape correcting section 13 detects the rotation amount of the distal end portion 2a1 as 01.

Next, the organ model shape correcting section 13 detects a second rotation amount θ2 of the distal end portion 2a1 of the insertion portion 2a between two times, between which the first rotation amount θ1 was detected, based on an output of the distal end position detecting apparatus 5 that is acquired from the organ model generating section 12, as shown in Column B of FIG. 16 (step S42).

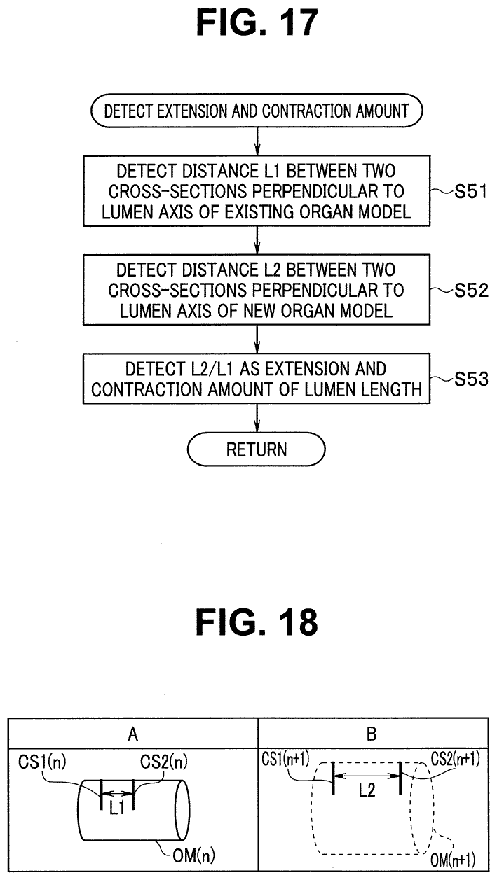

Further, the organ model shape correcting section 13 detects a difference (θ1-θ2) between the first rotation amount θ1 and the second rotation amount θ2 as the rotation amount of the organ (step S43), and returns to the processing of FIG. 11. FIG. 17 is a flowchart showing processing of detecting an extension and contraction amount in step S23 of FIG. 11 in the second embodiment. FIG. 18 is a chart for explaining the processing of detecting the extension and contraction amount in the second embodiment.

The organ model shape correcting section 13 selects two cross-sections CS1(n) and CS2(n) including feature points and perpendicular to the lumen axis in the existing organ model OM(n), as shown in Column A of FIG. 18, and detects a distance L1 between the two cross-sections CS1(n) and CS2(n) (step S51).

Next, the organ model shape correcting section 13 searches for two cross-sections CS1(n+1) and CS2(n+1) including the feature points and perpendicular to the lumen axis in the new organ model OM(n+1), which correspond to the two cross-sections CS1(n) and CS2(n), between which the distance L1 was detected, as shown in Column B of FIG. 18, and detects a distance L2 between the two cross-sections CS1(n+1) and CS2(n+1) (step S52).

Then, the organ model shape correcting section 13 sets the ratio of the distance L2 to the distance L1 (L2/L1) as the extension and contraction amount of the lumen diameter (step S53), and returns to the processing of FIG. 11. Note that when the ratio (L2/L1) is greater than 1, the lumen length extends, while the ratio (L2/L1) is smaller than 1, the lumen length contracts.

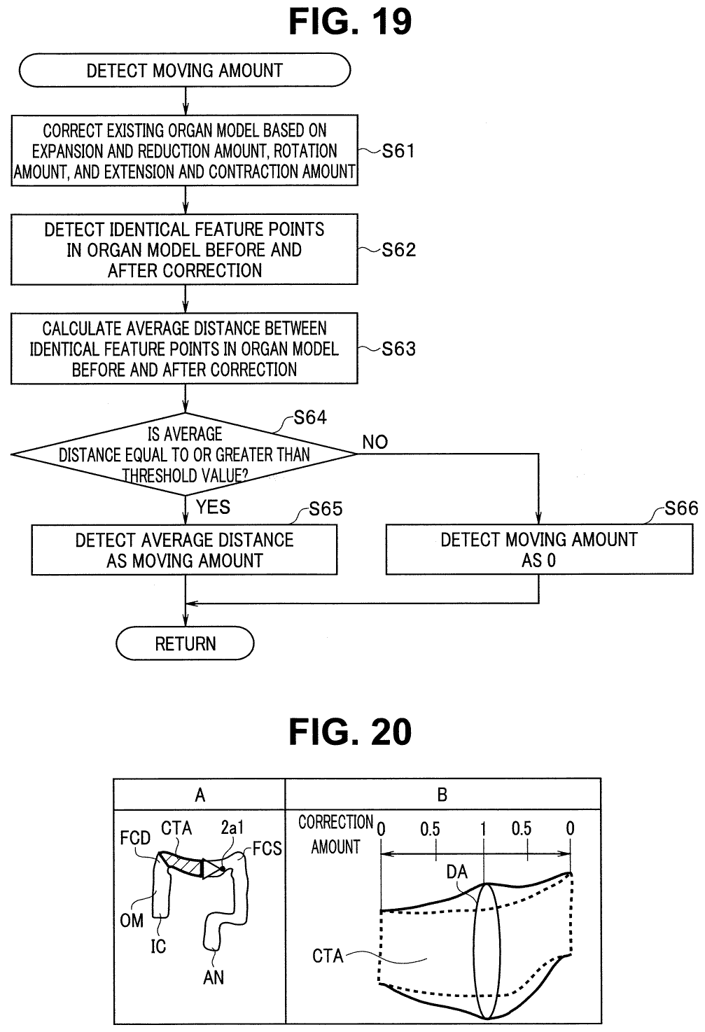

FIG. 19 is a flowchart showing processing of detecting a moving amount in step S24 of FIG. 11 in the second embodiment.

The organ model shape correcting section 13 corrects the existing organ model OM(n) based on the expansion and reduction amount detected in step S21, the rotation amount detected in step S22, and the extension and contraction amount detected in step S23 (step S61).

Next, the organ model shape correcting section 13 detects an identical feature point in the organ model before and after correction (step S62). Herein, the number of feature points to be detected may be one, but a plurality of feature points are preferable. Thus, an example of detecting a plurality of feature points will be described below.

Subsequently, the organ model shape correcting section 13 calculates an average distance between the plurality of identical feature points in the organ model before and after correction (step S63). Note that when the number of the feature points to be detected in step S62 is one, the processing of step S63 may be omitted, and the distance between the identical feature point in the organ model before correction and the identical feature point in the organ model after correction may be regarded as the average distance.

Then, the organ model shape correcting section 13 determines whether the calculated average distance is equal to or greater than a predetermined threshold value (step S64).

Herein, when the calculated average distance is determined to be equal to or greater than the threshold value, the average distance calculated in step S63 is detected as the moving amount (step S65).

In step S64, when the calculated average distance is determined to be less than the threshold value, the moving amount is detected as 0 (step S66). In other words, to prevent erroneous detection, when the average distance is less than the threshold value, it is determined that there is no movement of the organ.

When the processing of step S65 or step S66 is performed, the step then returns to the processing of FIG. 11.

FIG. 20 is a chart for explaining an example of a method for correcting the shape of the organ model in step S3A of FIG. 8 in the second embodiment.

The correction of the shape of the organ model is performed based on a change amount of the organ model detected in step S12.

A correction range at this time may be, for example, fixed distance ranges (portions of the organ model including a change site) in the front and the rear along the lumen axis, on the basis of the area (change site) that is a target for detection of the change amount. Herein, the fixed distance on the front side of the change site and the fixed distance on the rear side of the change site along the lumen axis may be the same distance or different distances.

The correction range may be a range (a portion of the organ model including a change site) having, as an end point, at least one of a landmark or the position of the distal end portion 2a1 of the insertion portion 2a. Herein, the landmark when the organ is a large intestine includes the intestinal cecum IC or the anal AN that is the end of the organ, the hepatic flexure FCD or the splenic flexure FCS that is a boundary between a fixed portion and a movable portion, and the like. The landmarks differ in accordance with the organ and are detectable by AI site recognition. In this manner, the organ model shape correcting section 13 can set a range outside a correction target in the organ model based on the organ type. The organ model shape correcting section 13 calculates the change amount by referring to the type information of the specific target in accordance with the organ type.

Alternatively, the organ model shape correcting section 13 may set the whole of the organ model as the correction range.

The correction amount within the correction range is controlled, for example, in accordance with the distance along the lumen axis, with the correction amount in an area that is the target for detection of the change amount as 1 and the correction amount at the end points of the correction range as 0.

Column A of FIG. 20 shows an example in which a correction range CTA is set by having, as opposite end points, the position of the distal end portion 2a1 of the insertion portion 2a present in a center portion of the transverse colon TC and the hepatic flexure FCD.

Column B of FIG. 20 shows an example in which in the correction range CTA, the correction amount is controlled in accordance with the distance along the lumen axis, with the correction amount in an area DA (a specific example is a fold) that is the target for detection of the change amount as 1 and the correction amounts at the opposite end points as 0. In other words, the organ model shape correcting section 13 reduces the correction amount in the shape of the organ model as the distance from the area DA (specific target) that is the target for detection of the change amount is increased.

According to such a second embodiment, the advantageous effects that are substantially the same as the advantageous effects of the aforementioned first embodiment are produced, and as shown in FIG. 10, the unobserved area UOA can be presented at a correct position.

Further, the change amount of the organ model can be detected using a method suitable for each of the expansion and reduction, rotation, extension and contraction, and movement.

Moreover, the correction amount is controlled in accordance with the distance along the lumen axis, so that the organ model after correction can be formed in an appropriate shape.

### Third Embodiment

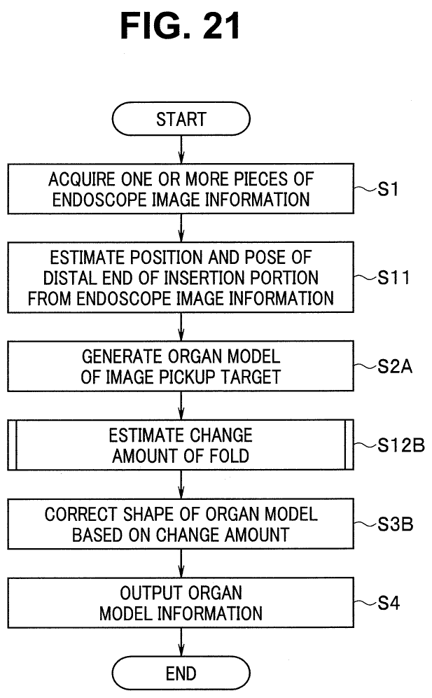

FIG. 21 to FIG. 39 show a third embodiment of the present invention, and FIG. 21 is a flowchart showing processing of the image processing apparatus 4 of the third embodiment. In the third embodiment, for the portions that are the same as the portions of the first and the second embodiments, the same reference signs are assigned and the descriptions will be omitted, as appropriate, and different points will be mainly described.

Upon starting the processing shown in FIG. 21, the processing of step S1 is performed to acquire the latest one or more endoscope images and the processing of step S11 is performed to estimate, from the endoscope images, the position and the pose of the distal end portion 2a1 of the insertion portion 2a.

Next, the processing of step S2A is performed to generate an organ model of an image pickup target. At this time, as shown in Column B of FIG. 6, when there is no unobserved area, the portion of the existing organ model denoted by the dotted line may not be retained in the memory 14 or may not be displayed on the monitor 8, which is the same as in the first and the second embodiments.

Subsequently, the organ model shape correcting section 13 estimates a change amount of a fold (specific target) of an intestine in the organ model (step S12B). There are some cases in which when the position or the shape of the organ changes, the feature points cannot be associated between the existing organ model and the new organ model. By contrast, in the folds of the luminal organ, neither the number of the folds nor the order relation of the folds changes, even when the position, the shape, or the like of the organ changes. Thus, in the present embodiment, the change amount of the organ model is surely estimated using the folds.

Further, the organ model shape correcting section 13 corrects the shape of the existing organ model based on the change amount of the fold in the estimated organ model (step S3B).

Thereafter, the processing of step S4 is performed to output the information on the corrected organ model to the monitor 8 or the like.

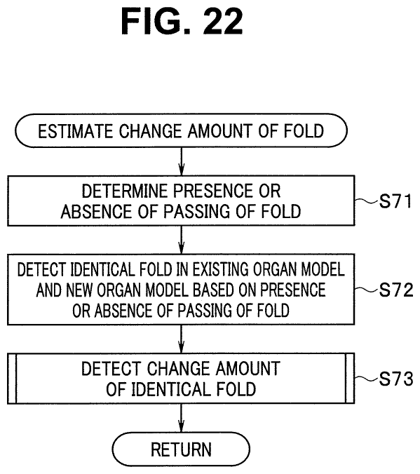

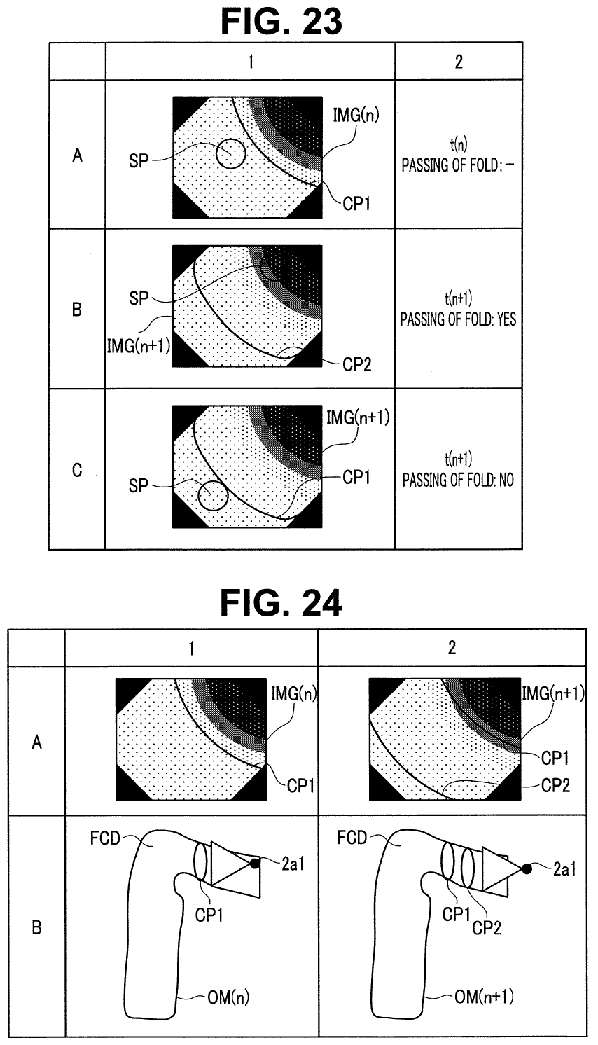

FIG. 22 is a flowchart showing processing of estimating a change amount of a fold in step S12B of FIG. 21 in the third embodiment. FIG. 23 is a chart for explaining processing of detecting the presence or absence of passing of the fold in the third embodiment.

The organ model shape correcting section 13 acquires the endoscope image IMG(n) at time t(n) in the past as shown in Column A1 of FIG. 23 and the endoscope image IMG(n+1) at the latest time t(n+1) as shown in Column B1 or Column C1 of FIG. 23. The endoscope image IMG(n) is an image used for generating an existing three-dimensional organ model and the endoscope image IMG(n+1) is an image used for generating a new three-dimensional organ model.

The organ model shape correcting section 13 searches the endoscope image IMG(n) and the endoscope image IMG(n+1), which are picked up at different times, for a common feature point SP other than the fold, as a tracking point.

Next, the organ model shape correcting section 13 determines whether the distal end portion 2a1 of the insertion portion 2a has passed a fold CP1 positioned on a far side near the feature point SP in the endoscope image IMG(n). Since Column A of FIG. 23 relates to time t(n), the passing of the fold is not determined, as shown in Column A2.

It is assumed that the endoscope image IMG(n+1) is as shown in Column B1 of FIG. 23. Thus, it is recognized that a fold CP2 on a proximal side relative to the feature point SP is a fold different from the fold CP1 positioned on the far side relative to the feature point SP. Therefore, the organ model shape correcting section 13 determines that the fold CP1 has been passed as in Column B2 of FIG. 23.

Meanwhile, it is assumed that the endoscope image IMG(n+1) is as shown in Column C1 of FIG. 23. In this case, it is recognized that the fold CP1 positioned on the far side near the feature point SP is the same as the fold CP1 shown in Column A1. Therefore, the organ model shape correcting section 13 determines that the fold CP1 is not passed as in Column C2 of FIG. 23.

In this manner, the organ model shape correcting section 13 determines the presence or absence of passing of the fold (step S71).

Next, the organ model shape correcting section 13 detects an identical fold in the existing organ model and the new organ model based on the presence or absence of passing of the fold (step S72).

FIG. 24 is a chart for explaining a state of associating the fold in the endoscope image and the fold in the organ model in the third embodiment.

As described above, even when the shape of the organ changes, neither the number of the folds nor the order relation of the folds changes, and thus, the fold in the endoscope image and the fold in the organ model are associated by counting the number of the folds.

In FIG. 24, Column A1 shows the fold CP1 in the endoscope image IMG(n) and Column A2 shows the folds CP1 and CP2 in the endoscope image IMG(n+1).

In FIG. 24, Column B1 shows that the distal end portion 2a1 of the insertion portion 2a is present at a position where only the fold CP1 is observed in the organ model OM(n). Column B2 shows that the distal end portion 2a1 of the insertion portion 2a is present at a position where the folds CP1 and CP2 are observed in the organ model OM(n+1).

When the identical fold is detected in step S72, subsequently, the organ model shape correcting section 13 detects the change amount of the identical fold (step S73).

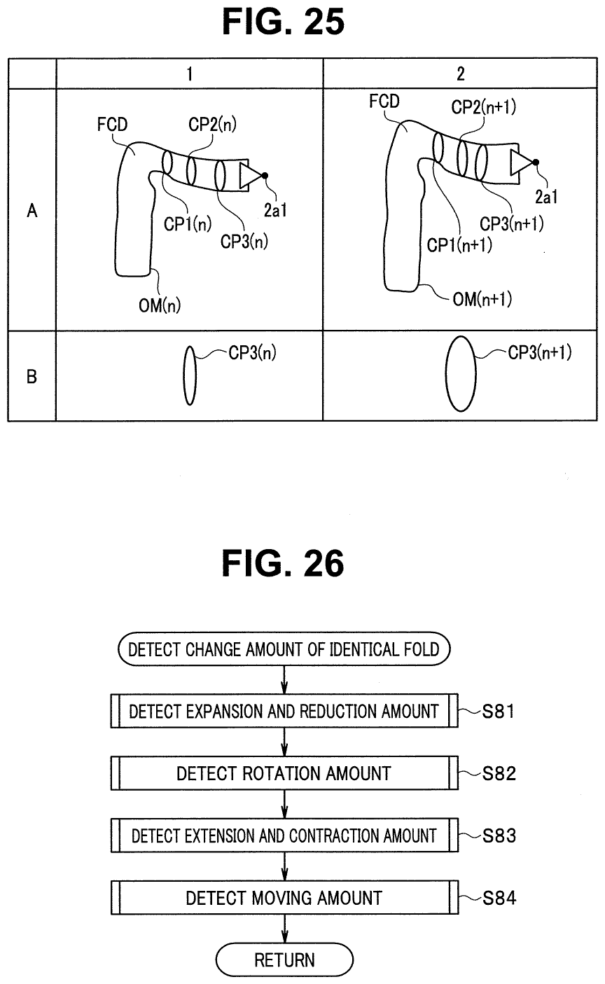

FIG. 25 is a chart for explaining a state of detecting a change amount of an identical fold in the third embodiment.

Column A1 of FIG. 25 shows a state in which three folds CP1(n), CP2(n), and CP3(n) are detected in the organ model OM(n) at time t(n).

Column A2 of FIG. 25 shows a state in which three folds CP1(n+1), CP2(n+1), and CP3(n+1), which respectively correspond to the three folds CP1(n), CP2(n), and CP3(n) of Column A1, are detected in the organ model OM(n+1) at time t(n+1).

Column B1 of FIG. 25 shows a state in which the fold CP3(n) of the three folds CP1(n), CP2(n), and CP3(n), which is the closest to the distal end portion 2al of the insertion portion 2a, is selected for detecting the change amount.

Column B2 of FIG. 25 shows a state in which the fold CP3(n+1) corresponding to the fold CP3(n) is selected for detecting the change amount.

The organ model shape correcting section 13 detects the change amount by comparing the fold CP3(n) shown in Column B1 of FIG. 25 and the fold CP3(n+1) shown in Column B2 of FIG. 25.

FIG. 26 is a flowchart showing processing of detecting the change amount of the identical fold in step S73 of FIG. 22 in the third embodiment.

The detection of the change amount of the identical fold by the organ model shape correcting section 13 is performed, for example, by detecting an expansion and reduction amount of the diameter of the identical fold in the new organ model relative to the fold in the existing organ model (step S81), detecting a rotation amount (step S82), detecting an extension and contraction amount between the two identical folds (step S83), and detecting a moving amount of the fold in a subject (step S84). Note that FIG. 26 shows an example of the detecting order, but the detecting order is not limited to the illustrated detecting order.

For performing the processing of detecting the expansion and reduction amount of the diameter in step S81 of FIG. 26, it is only necessary to, for example, detect the ratio D2/D1 of the distance between the corresponding feature points on the identical fold, in place of calculating the ratio D2/D1 of the distance between the feature points on the cross-section perpendicular to the lumen axis, which is described referring to FIG. 13 and FIG. 14. Alternatively, the exact description made referring to FIG. 13 and FIG. 14 may be applied.

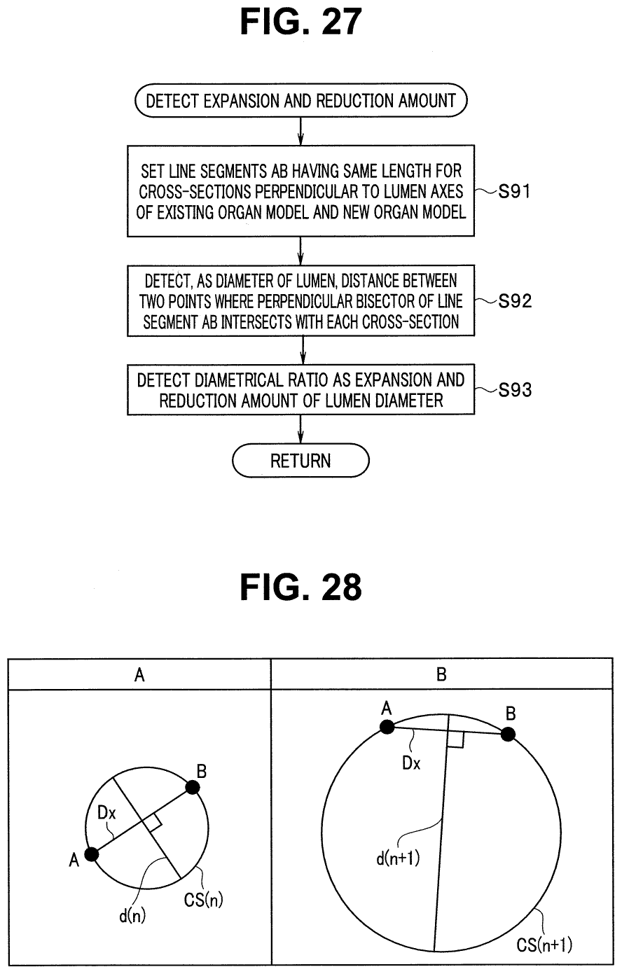

FIG. 27 is a flowchart showing another processing example of detecting an expansion and reduction amount of a diameter in step S81 of FIG. 26 in the third embodiment. FIG. 28 is a chart for explaining the other processing example of detecting the expansion and reduction amount of the diameter in the third embodiment.

Upon starting the processing shown in FIG. 27, the cross-sections CS(n) and the CS(n+1) perpendicular to the lumen axis are set in the existing organ model and the new organ model so as to include the identical feature point on the corresponding fold. Further, as shown in FIG. 28, line segments AB having the same length Dx are respectively set for the cross-sections CS(n) and CS(n+1) (step S91). At this time, at least one of the end points (for example, end point A) of the line segment AB may be set as the identical feature point on the corresponding fold.

Next, distances between two points where perpendicular bisectors of the line segments AB intersect with the cross-sections CS(n) and CS(n+1) are detected as diameters d(n) and d(n+1), respectively (step S92).

Then, a diametrical ratio d(n+1)/d(n) is detected as the expansion and reduction amount of the lumen diameter in the fold (step S93), and the step returns to the processing of FIG. 26.

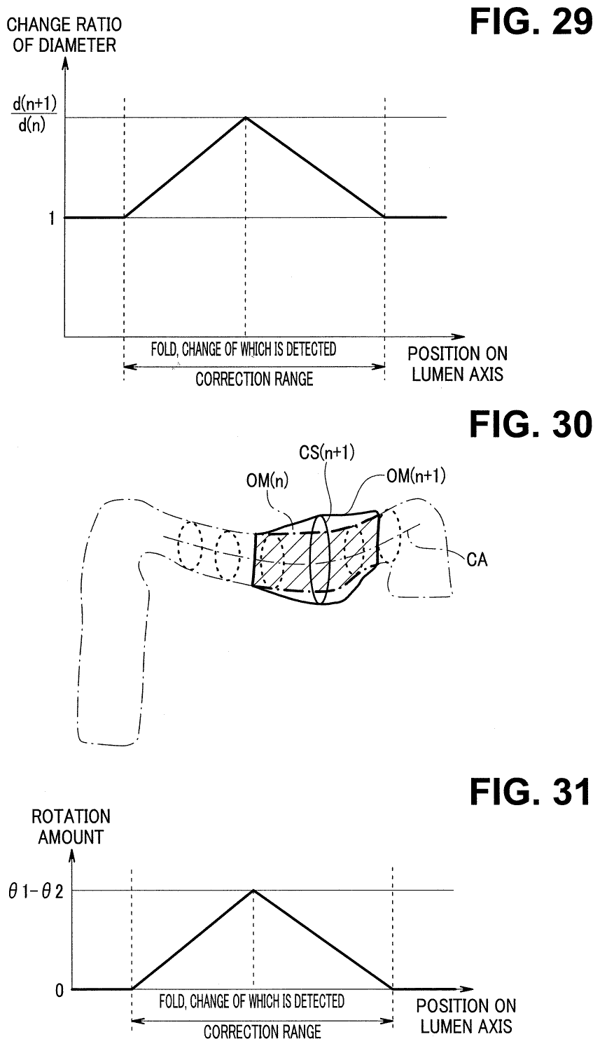

FIG. 29 is a graph for explaining an example of a method for correcting the expansion and reduction amount of the diameter of the organ model in the third embodiment. FIG. 30 is a view for explaining an example of correcting the expansion and reduction amount of the diameter of the organ model in a correction range in the third embodiment.

As described above in relation to FIG. 20, the correction of the expansion and reduction amount of the diameter may also be performed within the correction range including the fold, the expansion and reduction amount of which was detected. The correction range may be any of the fixed distance ranges in the front and the rear of the fold, a portion between two landmarks including the fold, and a portion between a landmark including the fold and the position of the distal end portion 2a1 of the insertion portion 2a, which is the same as described above. Further, the whole of the organ model may be the correction range, which is also the same as described above.

FIG. 29 shows a graph in which the change ratio of the diameter is set such that in the correction range along the lumen axis CA (see FIG. 30), the diameter is changed by the ratio d(n+1)/d(n) at a position of the fold, the expansion and reduction amount of the diameter of which was detected, and the change in the diametrical ratio at opposite end points of the correction range is set as 1. Thus, for example, at the midpoint between the position of the fold, the expansion and reduction amount of the diameter of which was detected, and the end points of the correction range, the change ratio of the diameter is {1+([{d(n+1)/d(n)}−1]/2)}. Note that the graph shown in FIG. 29 is one example, and the change ratio of the diameter may be formed in a curve.

In accordance with the expansion and reduction amount shown in FIG. 29, the organ model is radially corrected in the radial direction about the lumen axis, so that as shown in FIG. 30, the hatched correction range is corrected from the organ model OM(n) to the organ model OM(n+1). Note that in the ranges other than the correction target, there is no change between the organ model OM(n) and the organ model OM(n+1).

For the processing of detecting the rotation amount in step S82 of FIG. 26, for example, the descriptions made referring to FIG. 15 and FIG. 16 can be applied. At this time, in step S41, the detection of the first rotation amount θ1 based on the endoscope image may be performed by focusing on the fold in the endoscope image.

FIG. 31 is a graph for explaining an example of a method for correcting the rotation amount of the organ model in the third embodiment.

The rotation about the lumen axis of the luminal organ may also be corrected by setting a part or the whole of the organ model as the correction range, as with the diameter.

When the rotation amount is corrected, first, the lumen axis of the organ model in the correction range is estimated.

Next, in the correction range along the lumen axis, as shown in the graph of FIG. 31, the rotation amount is changed such that the rotation amount is changed by (θ1-θ2) at a position of the fold, the rotation amount of which was detected, and the rotation amounts at the opposite end points of the correction range are set as 0. Note that the graph shown in FIG. 31 is one example, and the change in the rotation amount may be formed in a curve.

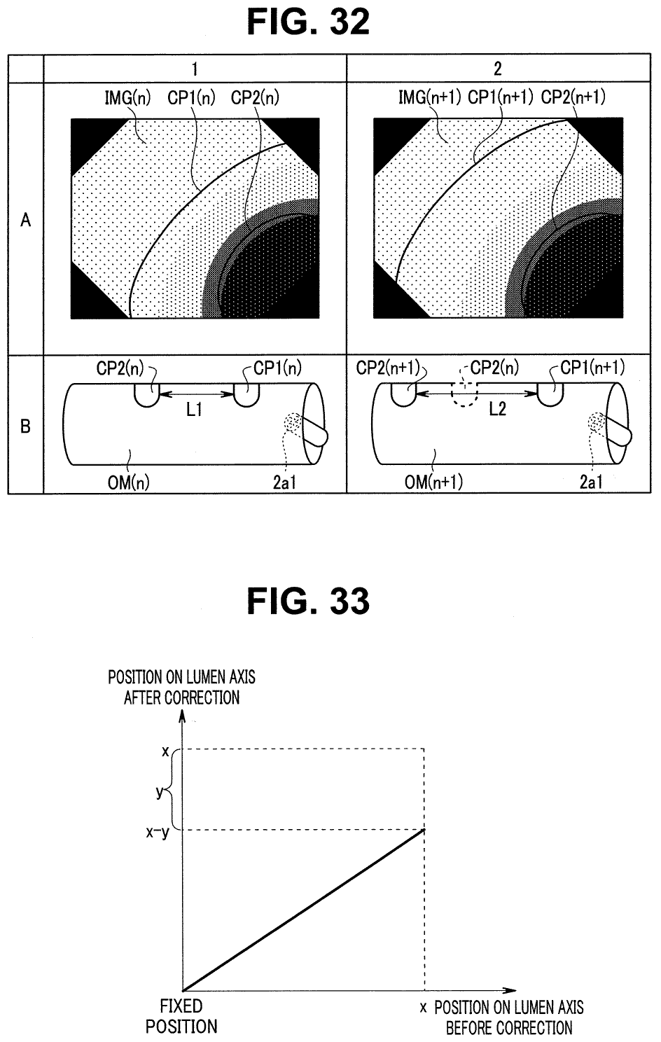

FIG. 32 is a chart for explaining processing of detecting the extension and contraction amount in step S83 of FIG. 26 in the third embodiment.

In FIG. 32, Column A1 shows the endoscope image IMG(n) picked up at time t(n) and Column A2 shows the endoscope image IMG(n+1) picked up at time t(n+1).

The organ model shape correcting section 13 detects two identical folds in the endoscope image IMG(n) and the endoscope image IMG(n+1) using, for example, AI. In the example shown in Column A of FIG. 32, the first fold CP1(n) and the second fold CP2(n) are detected in the endoscope image IMG(n) and the first fold CP1(n+1) and the second fold CP2(n+1) are detected in the endoscope image IMG(n+1).

When the distance between the folds at time t(n) in the past and time t(n+1) at present is changed, the extension and contraction amount is detected based on the depth value of the folds using, for example, the SLAM. Herein, it is assumed that changing of the distance L1 between the folds at time t(n) to the distance L2 at time t(n+1) is detected.

Then, the shape of the organ model is corrected such that the distance L1 between the folds in the existing organ model OM(n) as shown in Column B1 of FIG. 32 becomes the distance L2 shown in Column B2 of FIG. 32 in the new organ model OM(n+1).

For the extension and contraction of the luminal organ in the lumen axis direction also, as with the above, the correction of the extension and contraction amount can be performed within an appropriate correction range including the fold, the extension and contraction amount of which was detected. As an example, a portion between the landmark in the opposite direction of the last fold where the distal end portion 2a1 of the insertion portion 2a has passed and a fold that is the closest to the distal end portion 2a1 and where the distal end portion 2a1 of the insertion portion 2a has not passed yet may be set as the correction range.

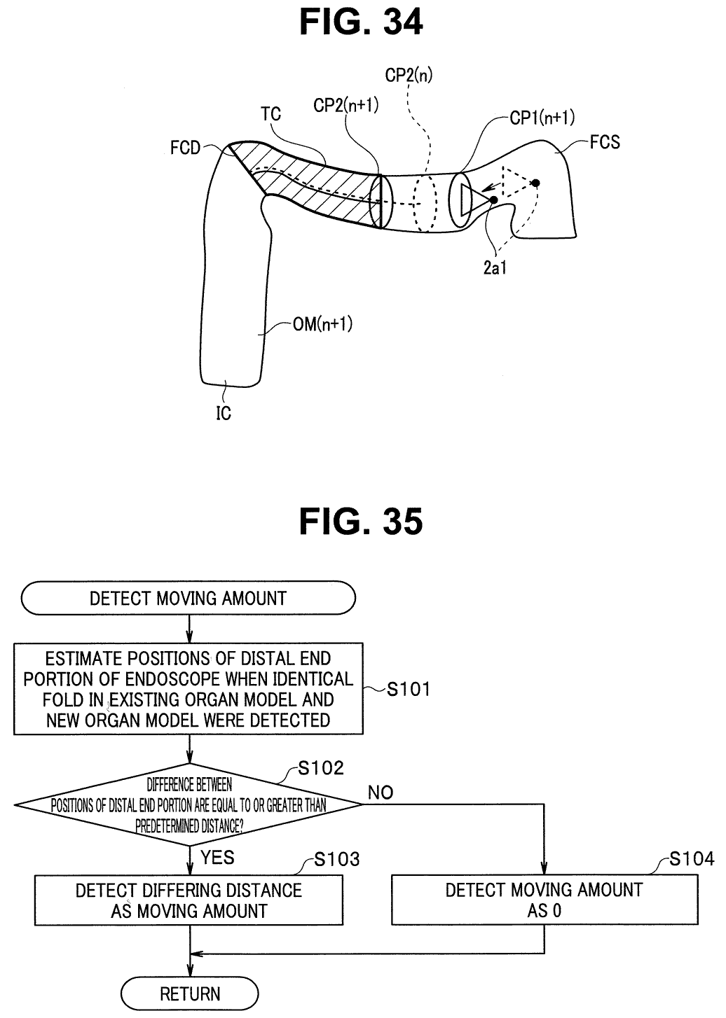

FIG. 33 is a graph for explaining an example of a method for correcting the extension and contraction amount of the organ model in the third embodiment. FIG. 34 is a view for explaining an example of correcting the extension and contraction amount of the organ model in the third embodiment.

The organ model shape correcting section 13, first, determines which portion of the organ model OM(n) is corrected, based on the moving direction of the distal end portion 2a1 of the insertion portion 2a. For example, in FIG. 34, it is assumed that the distal end portion 2a1 of the insertion portion 2a is moving from the splenic flexure FCS in a direction toward the hepatic flexure FCD. In this case, the organ model shape correcting section 13 sets the hepatic flexure FCD side (intestinal cecum IC side) in the transverse colon TC of the organ model OM(n) as the correction range as shown by hatching.

Next, the organ model shape correcting section 13 calculates a length×along the lumen axis from the hepatic flexure FCD as a landmark in the existing organ model OM(n) to the fold CP2(n), the change amount of which was detected.

Subsequently, the organ model shape correcting section 13 sets the hepatic flexure FCD, which is a landmark, as a fixed position, and calculates the extension and contraction amount from the fixed position to the fold CP2(n+1) at time t(n+1), herein, a reduced length y, for example, based on the change of the distance between the folds from L1 to L2. Thus, it is recognized that the length from the landmark to the fold CP2(n+1) along the lumen axis has become (x−y).

In this case, the extension and contraction ratio of the organ model is (x−y)/x. As shown in FIG. 33, the organ model shape correcting section 13 sets the landmark as the fixed position and corrects the shape of the organ model OM(n) such that the extension and contraction ratio of each point on the lumen axis is (x−y)/x. As a specific example, when x=10 and y=2, the extension and contraction ratio is (10−2)/10=0.8. Therefore, position 5 on the lumen axis having the fixed position as the origin becomes 5×0.8=4 after correction. Note that the graph shown in FIG. 33 is one example, and the change in the extension and contraction ratio may be formed in a curve.

By performing such correction, the organ model OM(n+1) as shown in FIG. 34 is calculated.

FIG. 35 is a flowchart for explaining processing of detecting the moving amount in step S84 of FIG. 26 in the third embodiment.

Upon starting the processing shown in FIG. 35, the positions of the distal end portion 2a1 of the insertion portion 2a when the identical fold in the existing organ model and the new organ model was photographed to be detected are estimated (step S101). The estimation of the position of the distal end portion 2a1 may be performed based on the endoscope image as described above or may be performed based on the information inputted from the distal end position detecting apparatus 5.

Next, it is determined whether the difference between the position of the distal end portion 2a1 when the fold was photographed in the existing organ model and the position of the distal end portion 2a1 when the fold was photographed in the new organ model is equal to or greater than a predetermined distance (step S102).

Herein, when the difference is equal to or greater than the predetermined distance, it is determined that the organ has moved, and the distance is detected as the moving amount (step S103).

In step S102, when the difference is less than the predetermined distance, it is determined that the organ has not moved, and the moving amount is detected as 0 (step S104). Herein, determining that the organ has moved only when the difference is equal to or greater than the predetermined distance is for the purpose of preventing erroneous determination due to a calculation error. When the processing of step S103 or step S104 is performed, the step then returns to the processing of FIG. 26.

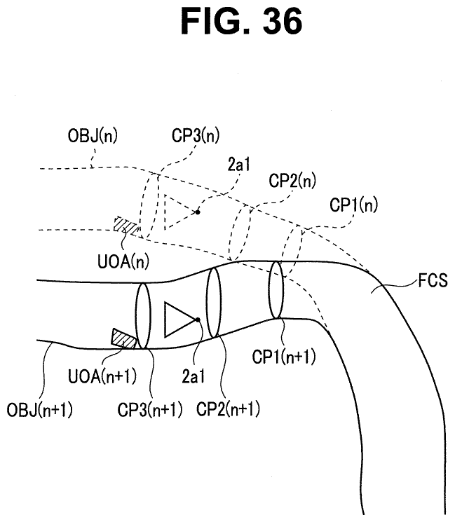

FIG. 36 is a view showing an example of detecting an identical fold in the existing organ model and the new organ model for determining movement of the organ in the third embodiment.

In FIG. 36, the dotted line indicates the position of the organ OBJ(n) of a subject before movement (at the first image pickup at time t(n)), and the solid line indicates the position of the organ OBJ(n+1) of the subject after movement (at the second image pickup at time t(n+1)). The identical fold at the first image pickup and the second image pickup is detected based on what number the fold is from the splenic flexure FCS as the landmark.

When the time proceeds from time t(n) to time t(n+1), the first fold CP1(n), the second fold CP2(n), and the third fold CP3(n) have moved to the position of the first fold CP1(n+1), the position of the second fold CP2(n+1), and the position of the third fold CP3(n+1), respectively, as counted in sequence from the splenic flexure FCS. The distal end portion 2a1 of the insertion portion 2a has passed the first fold CP1 and the second fold CP2 and is at a position facing the third fold CP3, and the third fold CP3 is closely observed in the endoscope image.

As viewed from the distal end portion 2a1 of the insertion portion 2a, unobserved areas UOA(n), UOA(n+1) are present on the far side near the third folds CP3(n), CP3(n+1). For the unobserved areas UOA, the unobserved area determining/correcting section 15 calculates the correct position at each time t(n), t(n+1), and displays the unobserved areas UOA on the monitor 8 or the like.

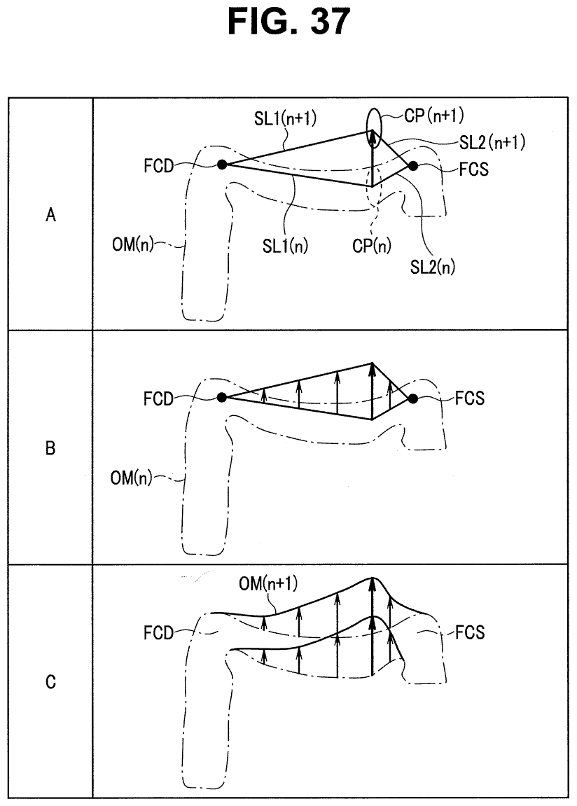

FIG. 37 is a view for explaining a method for correcting the shape of the organ model in accordance with the movement of the organ in the third embodiment.

The organ model shape correcting section 13 calculates the positions of the folds CP(n), CP(n+1) based on the position of the distal end portion 2a1 of the insertion portion 2a estimated in step S101, in correcting the shape of the organ model OM.

Next, the organ model shape correcting section 13 generates straight lines connecting the centers of the folds CP(n), CP(n+1), the movement of which was detected, and the center positions of the landmarks in the front and the rear of the folds CP(n), CP(n+1), as shown in Column A of FIG. 37. Specifically, for example, straight lines SL1(n), SL1(n+1) connecting the center position of the hepatic flexure FCD and each of the centers of the folds CP(n), CP(n+1) and straight lines SL2(n), SL2(n+1) connecting the center position of the splenic flexure FCS and each of the centers of the folds CP(n), CP(n+1) are generated.

Subsequently, the organ model shape correcting section 13 calculates, as the moving amount of each point, the distance from a predetermined point on the straight line SL1(n) to a predetermined point on the straight line SL1(n+1), as shown in Column B of FIG. 37, for example. The method of setting the points includes a method in which for example, the points where a surface perpendicular to a straight line connecting the center position of the hepatic flexure FCD and the center position of the splenic flexure FCS, and the straight lines SL1(n) and SL1(n+1) intersect with each other are set as the points.

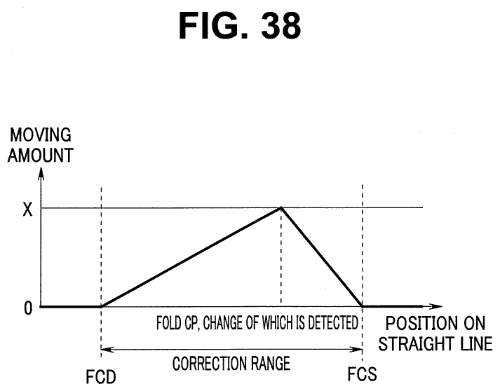

FIG. 38 is a graph for explaining the method for correcting the shape of the organ model in accordance with the movement of the organ in the third embodiment. As shown in FIG. 38, a portion between the hepatic flexure FCD and the splenic flexure FCS is the correction range, and when the moving amount of the fold CP, the change of which was detected, is X, as the position on the straight line is moved from the hepatic flexure FCD to the fold CP, the moving amount is monotonously increased, and as the position on the straight line is moved from the fold CP to the splenic flexure FCS, the moving amount is monotonously decreased. Note that the graph shown in FIG. 38 is an example, and the change in the moving amount may be formed in a curve.

Then, the organ model shape correcting section 13 corrects the portion between the hepatic flexure FCD and the splenic flexure FCS of the organ model OM(n) in accordance with the calculated distance as shown in Column C of FIG. 37, so as to calculate the corrected organ model OM(n+1).

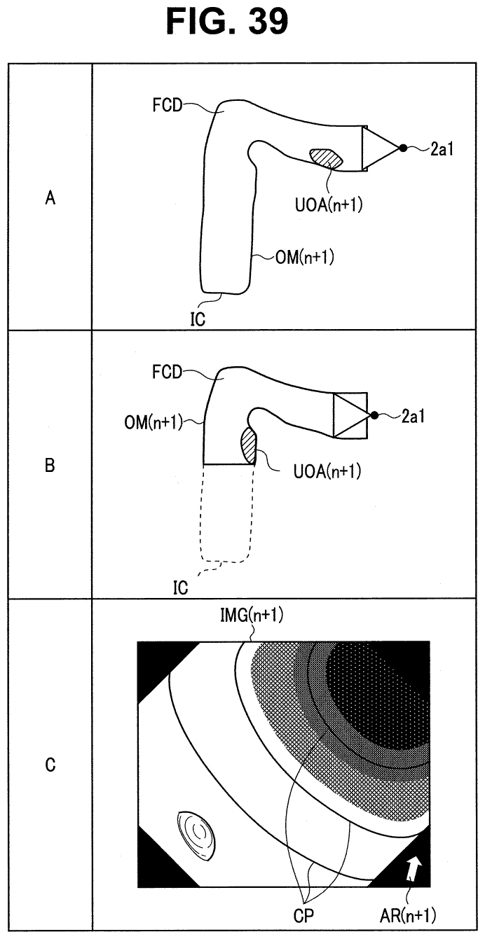

FIG. 39 is a chart showing an example of displaying the organ model and an unobserved area in the third embodiment.

When the shape of the organ model OM(n) is corrected and the organ model OM(n+1) is calculated, the organ model OM(n+1) after correction is displayed on the monitor 8.

Column A of FIG. 39 shows the organ model OM(n+1) after correction displayed on the monitor 8. In the organ model OM(n+1), the range from the distal end portion 2a1 of the insertion portion 2a through the hepatic flexure FCD to the intestinal cecum IC is displayed. At this time, an area for which the organ model OM(n+1) is not generated may be displayed as the unobserved area UOA(n+1). Further, in the organ model OM(n+1), the position of the distal end portion 2a1 of the insertion portion 2a and the direction of view are displayed using, for example, a triangle symbol. When the triangle symbol is used, one vertex of the triangle symbol indicates the position of the distal end portion 2a1 and two sides sandwiching the vertex indicate the direction of view and the range of view. Other symbols and the like may also be used.

Column B of FIG. 39 shows an example of displaying the organ model OM(n+1) only at the position subsequent to the unobserved area UOA(n+1) in the moving direction of the distal end portion 2a1 of the insertion portion 2a. For example, it is assumed that the distal end portion 2a1 moves from the intestinal cecum IC to the hepatic flexure FCD, and is further moving from the hepatic flexure FCD toward the splenic flexure FCS. At this time, the organ model OM(n+1) before the position of the unobserved area UOA(n+1) may not be displayed, as shown by a dotted line.

Column C of FIG. 39 is an example of displaying the endoscope image IMG(n+1) on the monitor 8 and displaying, by an arrow AR(n+1), the direction from the distal end portion 2a1 toward the unobserved area. At this time, the distance from the distal end portion 2a1 to the unobserved area may be further displayed by means of the length (or the thickness, the color, or the like) of the arrow AR(n+1).

When the moving speed of the distal end portion 2a1 of the insertion portion 2a is greater than a predetermined threshold value, the image of the fold CP is not clearly picked up in some cases. In the present embodiment, the fold CP is used for correcting the organ model OM. Thus, when the image of the fold CP needs to be clearly picked up, the moving speed of the distal end portion 2a1 may be displayed on the monitor 8 so as to issue an alert using display, sound, or the like when the moving speed is equal to or greater than the threshold value.

According to such a third embodiment, the advantageous effects that are substantially the same as the advantageous effects of the aforementioned first and second embodiments are produced, and the identical fold can be detected by determining the presence or absence of passing of the fold, taking advantage of the fact that even when a change occurs in the organ, the order relation of the folds or the number of the folds is not affected. The change in the shape of the organ can be accurately estimated by detecting the presence or absence of a change and the change amount in the identical fold. Further, the direction and the position of an unobserved area after correction are displayed together with the organ model after correction or the latest endoscope image, so that the unobserved area is accurately presented, thereby preventing any lesion from being overlooked or the like.

Note that in each of the aforementioned embodiments, the shape of the organ model may be corrected based on the information acquired from the endoscope 2 or the peripheral equipment of the endoscope 2. Alternatively, the shape of the organ model may be corrected by combining the information acquired from the endoscope 2 or the peripheral equipment of the endoscope 2 and the endoscope image information.

For example, when air is fed to the inside of the organ from the endoscope 2, the organ inflates to change in shape. Thus, the shape of the organ model may be corrected by estimating the inflation amount of the organ based on the amount of air fed to the inside of the organ.

In the aforementioned description, the case in which the present invention is the image processing apparatus of the endoscope system has mainly been described, but the present invention is not limited to such an apparatus, and the present invention may be an image processing method for performing the same functions as the functions of the image processing apparatus, a program that causes a computer to perform the same processing as the processing of the image processing apparatus, a non-transitory recording medium (nonvolatile storage medium) that is readable by a computer and that stores the program, or the like.

Further, the present invention is not limited to the exact aforementioned embodiments, and can be embodied by modifying the constituent elements within the scope without departing from the gist of the present invention at the implementation stage. Furthermore, various aspects of the invention can be formed by appropriately combining a plurality of constituent elements disclosed in the aforementioned embodiments. For example, some constituent elements may be deleted from all the constituent elements shown in the embodiments. Moreover, the constituent elements across the different embodiments may be appropriately combined. Thus, it goes without saying that various modifications and applications are available within the scope without departing from the gist of the invention.

What is claimed is:

1. An image processing apparatus comprising a processor,
wherein
the processor is configured to:
after acquiring endoscope image information from an endoscope to generate an organ model,
continue acquiring the endoscope image information;
specify, based on latest endoscope image information, a change site of the organ model already generated;
correct a shape of at least a part of the organ model including the change site; and
output information on the organ model corrected.

2. The image processing apparatus according to claim 1, wherein the processor generates a new organ model based on the latest endoscope image information and corrects the shape of the organ model already generated, based on the new organ model.

3. The image processing apparatus according to claim 1, wherein the processor corrects a whole of the organ model.

4. The image processing apparatus according to claim 1, wherein the processor sets a range other than a correction target in the organ model based on an organ type.

5. The image processing apparatus according to claim 1, wherein the processor calculates, from a specific target included in a plurality of pieces of endoscope image information picked up at different times, a change amount of the specific target and corrects the shape of the organ model based on the change amount.

6. The image processing apparatus according to claim 5, wherein the processor calculates, as the change amount, at least one of an expansion and reduction amount, a rotation amount, an extension and contraction amount, or a moving amount.

7. The image processing apparatus according to claim 6, wherein the processor calculates, based on a change amount in a distance between a plurality of specific targets, at least one of the expansion and reduction amount or the extension and contraction amount.

8. The image processing apparatus according to claim 6, wherein the processor calculates a first rotation amount of the plurality of pieces of endoscope image information based on the specific target, acquires a second rotation amount of a distal end portion of an insertion portion of the endoscope from an external position detecting sensor, and calculates the rotation amount based on the first rotation amount and the second rotation amount.

9. The image processing apparatus according to claim 6, wherein the processor calculates the expansion and reduction amount, the rotation amount, and the extension and contraction amount, corrects the shape of the organ model already generated, based on the expansion and reduction amount, the rotation amount, and the extension and contraction amount, and thereafter, when a distance between the specific target in the organ model before correction and the specific target in the organ model after correction is equal to or greater than a threshold value, calculates, as the moving amount, the distance between the specific target in the organ model before correction and the specific target in the organ model after correction.

10. The image processing apparatus according to claim 5, wherein the processor acquires the endoscope image information from the endoscope by frame, and calculates the change amount by the frame.

11. The image processing apparatus according to claim 5, wherein the processor calculates the change amount referring to type information of the specific target.

12. The image processing apparatus according to claim 5, wherein the processor reduces a correction amount of the shape of the organ model as a distance from the specific target increases.

13. The image processing apparatus according to claim 5,
wherein
an organ as a target for generation of the organ model is an intestine, and
the specific target is a fold of the intestine.

14. An image processing method, comprising:
after acquiring endoscope image information from an endoscope to generate an organ model,
continuing acquiring the endoscope image information;
specifying, based on latest endoscope image information, a change site of the organ model already generated;
correcting a shape of at least a part of the organ model including the change site; and
outputting information on the organ model corrected.

15. A non-transitory storage medium that is readable by a computer and that stores a program, wherein
the program causes the computer to:
after acquiring endoscope image information from an endoscope to generate an organ model,
continue acquiring the endoscope image information,
specify, based on latest endoscope image information, a change site of the organ model already generated,
correct a shape of at least a part of the organ model including the change site, and
output information on the organ model corrected.
